(19) United States
(12) Patent Application Publication
(10) Pub. No.: US 2008/0051261 A1
(43) Pub. Date: Feb. 28, 2008

(54) Exercise protocols for treadmills and bicycle ergometers for exercise, diagnostics and rehabilitation

(76) Inventor: Charles A. Lewis, Carrabelle, FL (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/893,304

(22) Filed: Aug. 14, 2007

Related U.S. Application Data
(60) Provisional application No. 60/839,777, filed on Aug. 25, 2006.

(51) Int. Cl. A63B 22/02; A61B 5/00

(52) U.S. Cl. 482/54

(57) ABSTRACT

In exercise testing it is desirable to have an exercise protocol which helps to maximize the subject's ability to reach their peak workload. The exercise protocols taught here are designed to give exercise workloads which can be progressively graded and use natural cadences of walking and running to give a more comfortable pace at various workloads, and are performed over an adequate time. A lower extremity ergometry protocol method is also taught which gives a more accurate workload, and can help to minimize stress on the knees. This patent also teaches that ramping or workload for exercise protocols may be controlled by the physiologic response to the exercise. These protocols are also useful in exercise training and in rehabilitation.

The present application claims the benefit and priority of U.S. Provisional Patent Application Ser. No. 60/839,777, filed on Aug. 25, 2006. The entire content of this application is hereby incorporated by reference herein.

### BACKGROUND

There are many exercise protocols for stress testing and several of these have been summarized by Fletcher. It has been demonstrated that exercise protocols with smaller steps in work gradient allow subjects to attain a higher level of exercise and that these test have more reproducible results and allow the subject to reach higher workloads. The Bruce Treadmill Protocol, which remains the most recognizable to clinicians and is still in common use, is an incremental protocol with uneven steps of 2-3 metabolic equivalents (METs). The steps are spaced 3 minutes apart, and are not equal in the workload increments. Other commonly used treadmill protocols have smaller steps, often 2 minutes apart, but while the increments may be equal steps in speed or gradient, they are not equal steps with respect to workload for the subject being tested.

Exercise workload level during stress testing is often expressed in METs or, if directly assessed by ventilatory expired gas, oxygen consumption (\dot{V}O₂ in mlO₂·kg⁻¹·min⁻¹). One MET is a measure of the basil metabolic rate, with higher MET levels achieved during exercise being multiples of this. One MET is approximately equivalent to 3.5 mlO₂·kg⁻¹·min⁻¹, and is based on an average for a healthy 70 kg 40 year old male.

It has been shown by Smokler that the reproducibility of exercise testing to determine work load which caused angina was inversely related to the duration of exercise. If the work rate increase was too steep, the reproducibility fell. In a given patient, test duration less than ten minutes had high levels of variance for the same level of chest discomfort. Gains in reproducibility diminished as the test length increased towards 20 minutes.

Buckfuhrer used stepped protocols in healthy subjects to determine protocols which allowed for achieving maximal work rates. He found that the \dot{V}O₂ₘₐₓ (the highest sustained level of oxygen consumption that the subject is able to achieve), was significantly higher on tests where the increment in work magnitude resulted in a test duration of 8 to 17 minutes. This study, however, may have had limited maximal workload levels achieved because it used a stepped protocol, which is known to give lower a \dot{V}O₂ₘₐₓ than protocols with a continuous exercise ramp.

A test duration which allows the highest attainable workload and provides high reproducibility of results is desirable. Most experts currently recommend a work load which ramps to the subject's predicted maximal workload over an 8 to 10 minute period. However, reading of the referenced cited in these 8-10 minute recommendations begs the question of how the 8 to 10 minute recommendation was formed. Certainly, Smokler's data suggest a minimum of 10 minutes, and was not for \dot{V}O₂ₘₐₓ, but for angina which would be expected to occur at a lower workload.

An exercise test must allow sufficient time to recruit the metabolic processes in the muscles required to increase to the workload demanded. This process may be measured indirectly by the change in the amount of \dot{V}O₂ₘₐₓ for the given change in work rate. The time constant of \dot{V}O₂ reflects the time required for the pulmonary, cardiovascular and skeletal muscle systems to adjust to a change in workload. This process includes the time for the oxygen uptake in the muscle cell as well as the time it takes to detect this uptake at the measuring site, which for pulmonary exercise testing is the lung ventilation. The O₂ uptake/work rate slope in healthy subjects is normally about 10.2 ml O₂/minute/watt and is fairly independent of sex, age, or height. The slope remains the same for obese patients. The Δ\dot{V}O₂/ΔWR declines with age and in patients with coronary artery disease (CAD), congestive heart failure (CHF) and with deconditioning. A fall in the Δ\dot{V}O₂/ΔWR slope during exercise (beyond the anaerobic threshold) may be used as a test of myocardial ischemia.

Another weakness with traditional treadmill testing is that human ambulation is not equally efficient at different speeds and different cadences. Thus a constant increase in speed or in slope does not guarantee a constant increase in work performed by the subject. This is also true for cycle ergometry. The present invention is a method for treadmill and ergometer exercise protocols designed to give workloads more even increases in work throughout the exercise.

Yet another weakness with traditional exercise testing is that it often paces the person exercising (speed and/or grade) at levels which are awkward and unnatural. This can not only cause non-linear steps in workload, but may cause the subject to feel unstable during ambulation. To stabilize themselves, subjects often take a hold of the hand rails, which has the effect of lowering the subject's actual \dot{V}O₂ for a given workload, creating an inability to accurately determine the metabolic cost of exercise by treadmill speed and grade. An uncomfortable exercise pace may also contribute to a patient asking to terminate the test early, also leading to a loss of diagnostic information, and invoking the need to do another test, adding costs and risks.

### SUMMARY

The present disclosure relates to a method for a progressive exercise protocol for use with a treadmill. This method for exercise protocols provides for small steps or a continuous gradient in workloads over an adequate time for recruitment of the metabolic processes required to approach or achieve maximum workloads for the subject. This method uses the natural cadence of ambulation in order to give more effective and more comfortable exercise especially as it relates to exercise testing and rehabilitation.

The present disclosure also relates to method for a progressive exercise protocol for use with cycle ergometers.

The present disclosure also relates to the method of using the physiologic response to exercise to control the rate of increasing the workload.

The present disclosure also relates to the use of one or more hand holds, for use during exercise, which are designed to minimize the transfer of the subject's weight to the hand hold during exercise.

A reasonable exercise ramp can be predicted for a subject by taking the difference of resting metabolic cost, and the subjects predicted maximum exercise output, and creating a slope for this increase in work over an appropriate time period. Several published algorithms are available for prediction of \dot{V}O₂ₘₐₓ.

Malatesta demonstrated that metabolic cost of standing is equivalent to about 2 METs. Thus standing “rest” is an important metric in testing, especially for those with low expectations, i.e., those with symptoms of CHF, who often had a peak work level of under 5 METS. The patient may also be seated prior to exercise to give a lower baseline level.

The treadmill protocol may then go from the minimum exercise work load seated; at less than 2 METS, or standing; about 2 METS or about 7 mlO₂·kg⁻¹·min⁻¹ for standing, to the predicted maximum exercise capacity as a continuous ramp or as frequent small ramped steps of no more than about one minute. Workload at any point during the test can thus be estimated as:
Workload=(MECp−Bw)×Time/Duration+Bw

Where
Workload: Preferably in O₂ cost in mlO₂·kg⁻¹·min⁻
MECp=Maximum Predicted Exercise Capacity; preferably in O₂ cost in mlO₂·kg⁻¹·min⁻¹
Bw=Beginning Workload; (resting workload) preferably in O₂ cost in mlO₂·kg⁻¹·min⁻¹
Time=Time elapse since onset of exercise
Duration=Targeted exercise test duration

Workload for this equation should be done in metabolic cost rather than mechanical workload, as metabolic costs includes the effect of exercise efficiency and inefficiencies. This is because not all work done results in mechanical energy, and the relationship of metabolic work to mechanical output may not be linear.

Cardiopulmonary stress test expected duration should be at least 8 minutes for a test of \dot{V}O₂ₘₐₓ, and preferably longer. Thus, in the example of a patient with CHF and a predicted maximum O₂ uptake of 17.5 mlO₂·kg⁻¹·min⁻¹ (5 METs), a beginning workload of 8.75 mlO₂·kg⁻¹·min⁻ (2.5 METs), and a selected test duration of 10 minutes, the workload would increase by 0.875 ml mlO₂·kg⁻¹·min⁻¹. The work rate at the anaerobic threshold would be expected to be close to 13.5 mlO₂·kg⁻¹·min⁻¹ at about 5 and a half minutes.

A target test duration of ten minutes is appropriate for most older subjects or subjects with a predicted maximum workload of less than 35 mlO₂·kg⁻·min⁻¹ (10 METs). For subjects with a higher predicted workload a longer test is advised. A simple general principle is to use a target test duration of at least 1 minute per MET predicted maximum workload, using a minimum targeted exercise duration of 10 minutes. The phrase “target test duration” is used as some patients may not complete the test due to angina, leg pain, arrhythmia or other factors.

A limitation to the use of treadmills is that ambulation has natural speeds or cadences, and walking or running outside of those speeds may feel awkward or uncomfortable to the individual exercising. Existing treadmill protocols ignore these natural rhythms. There are certain speeds which are faster than a walk and slower than a run, and therefore not typical of the natural ambulation. These speeds are uncomfortable for most subjects, and may contribute to the use of hand rails, which affect the work load performed by the patient causing the test results to be inaccurate. Many protocols begin with a speed which is uncomfortably slow, and often pair this with a slope to increase the work load. Others such as the Balke-Ware protocol use a set speed and varies the slope, which may give some appropriate cadences, but still is not optimized for patient comfort.

Lower extremity ergometer protocols have been based on the mechanical workload generated (often expressed in Watts or kilogram/meters/minute) rather than the subjects metabolic work rate used to generate this work. This approach may give a work slope which is not consistent across the test. In older subjects who may have the combination of a low exercise capacity and arthritic joints in their lower extremities, much of the workload may be produced by crank speed alone, thus creating much less strain on the knees.

### BRIEF DESCRIPTION OF THE ILLUSTRATIONS

Reference will now be made to the accompanying drawing figures.

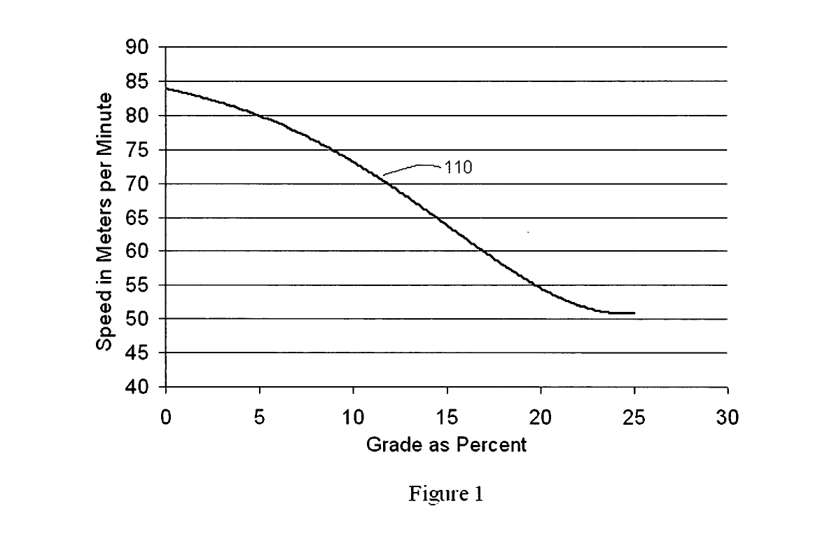

FIG. 1 is a graph showing typical self selected walking speeds for a normal adult human.

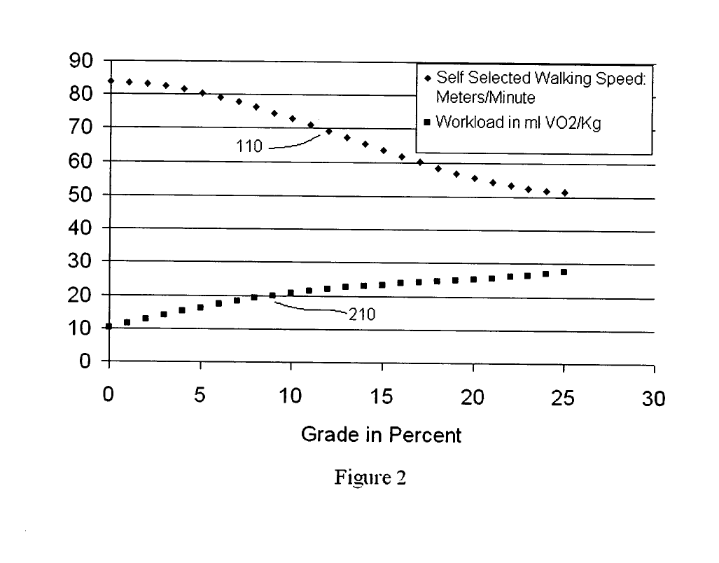

FIG. 2 is a graph showing the workload in mlO₂·kg⁻¹·min⁻¹ at various speeds and grades for the typical adult self selected walking speeds (SSWS).

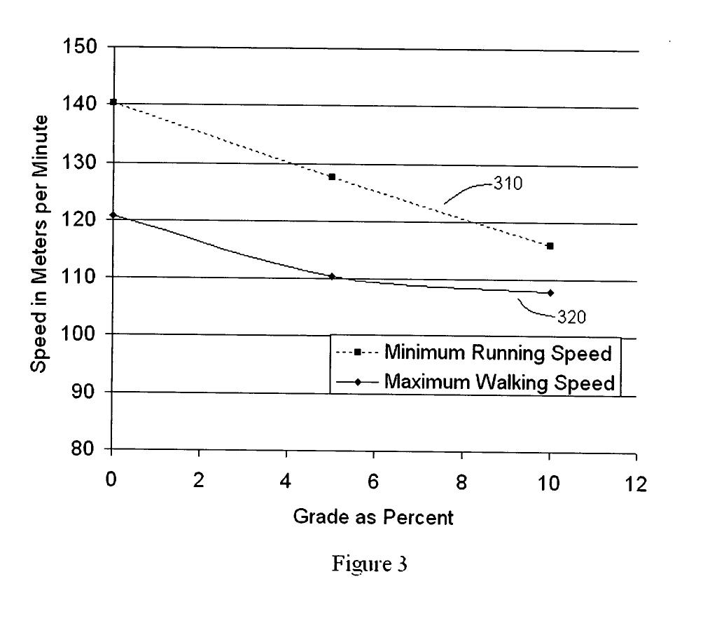

FIG. 3 is a graph illustrating the maximum walking speed and minimum running speed for healthy adults.

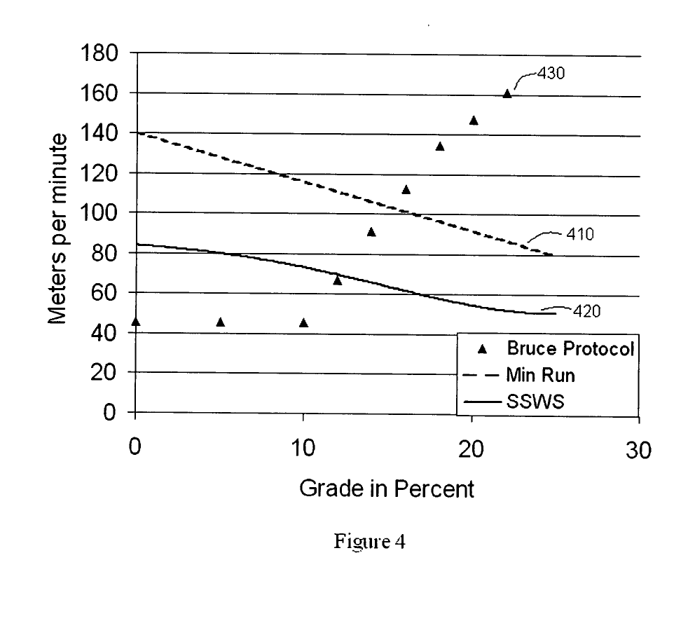

FIG. 4 is a graph showing typical self selected walking speeds, and minimal comfortable running speeds for different grades and the steps of the Bruce Protocol.

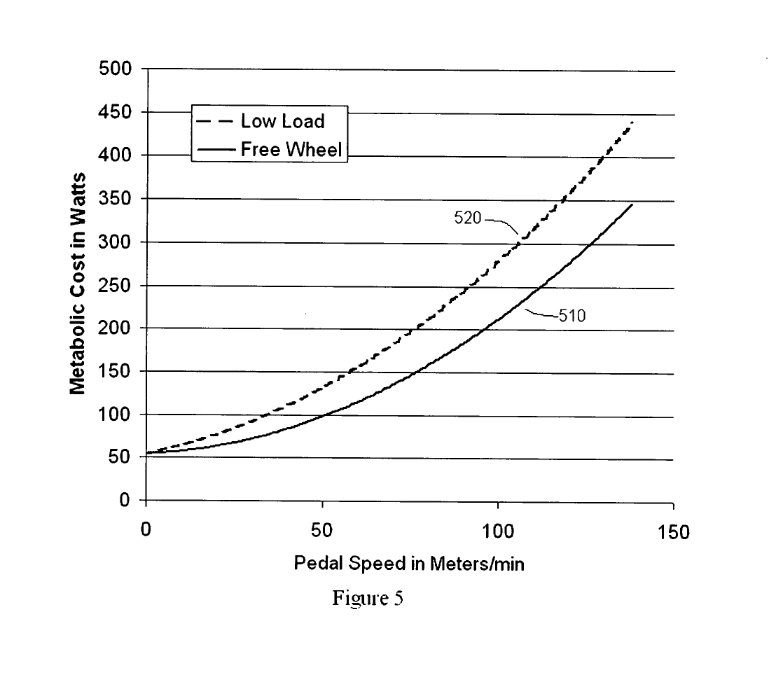

FIG. 5 is a graph showing the metabolic cost in Watts for lower extremity ergometry for freewheeling, and the metabolic cost with a low load.

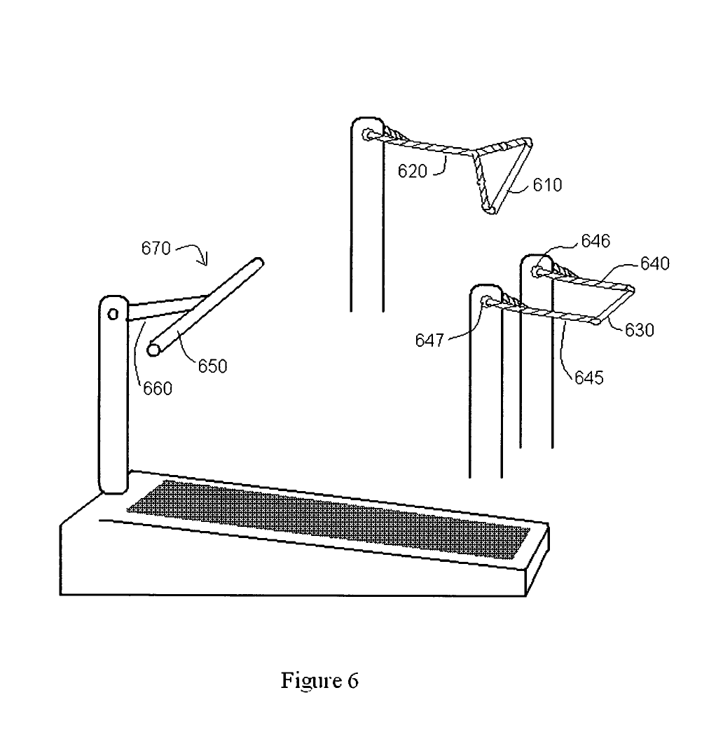

FIG. 6 shows drawings for hand holds for treadmills which preventing the person exercising from unloading their weight during exercise.

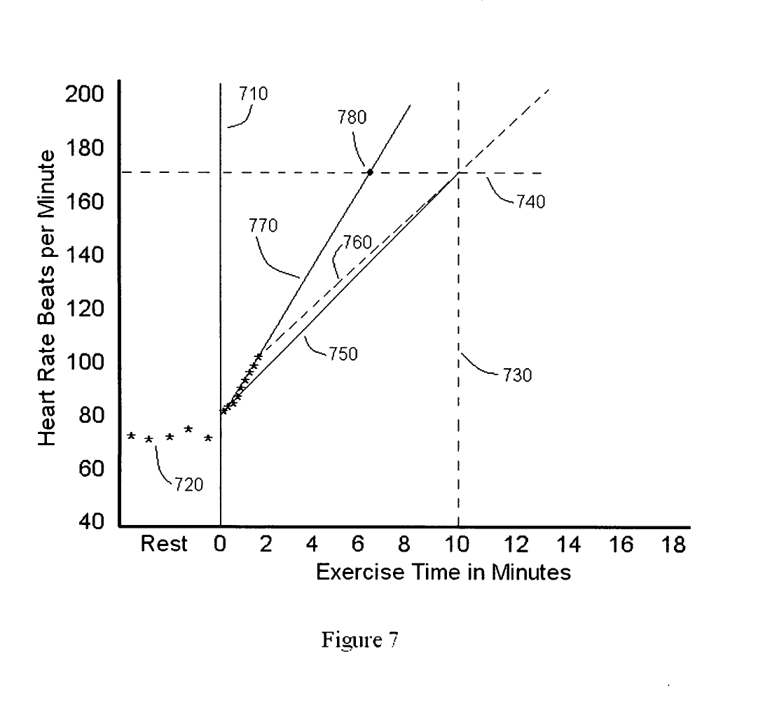

FIG. 7 illustrates a simplified example of how the workload ramp speed may be adjusted during a test according to the increase of heart rate (HR) as and example of controlling the workload from physiologic measures during testing.

### DETAILED DESCRIPTION

The present inventions now will be described with reference to the accompanying drawings, in which some, but not all embodiments of the inventions are shown. Indeed, these inventions may be embodied in many different forms and should not be construed as limited to the embodiments set forth herein. Rather, these embodiments are provided so that this disclosure will satisfy applicable legal requirements.

Treadmill Protocol

The present invention is a treadmill protocol which uses natural velocities of ambulation which are more comfortable for walking on a level surface and on incline slopes. This protocol is designed so that it may be used with microprocessor or computer controlled treadmills.

Minetti has shown that certain walking speeds are most comfortable for a given slope. These allow combinations of speeds and slopes which are comfortable for most subjects. Using these speed/slope relationships, workload calculations have been prepared at various levels. The workload is not linear because ambulation does not have equal efficiency at different speeds and slopes.

FIG. 1 shows polynomial curve 110 for the association of comfortable walking speed and slope (grade×100). The Y axis shows the typical self-selected speed in meters per minute and, and the X-axis shows the grade in percent. It can be seen that as the grade increases from level ground (grade=0) to a steeper slope, the pace which is comfortable decreases. This is explained by the progressive loss of the inverted pendulum motion which gives efficiency in ambulation.

Typically older patients and patients with heart disease may not run on the treadmill. They are often concerned with falling or tripping, and may be limited by arthritic conditions. Many subjects are deconditioned, and may not have run in many years. It is important to have a treadmill program suitable to persons who cannot run.

The present invention makes use of the most comfortable speeds for a given slope. These typically also give a speed close the maximum efficient for ambulation at those slopes. This exercise protocol contains an inverse speed slope progression (ISSP) in which the speed slows as the slope increases for at least a part of the exercise workload ramp.

FIG. 2 shows typical adult self selected walking speeds (SSWS) according to gradient 210, and workload 220. The chart has the grade of the slope on the X-axis. The Y-axis at the top part of the slope gives the self selected walking speed in meters per minute, and below the Y-axis gives the workload in ml O₂/kg for the corresponding values of grade and speed. Thus, at a grade of zero and a SSWS of about 84 meters per minute gives a workload of about 10 ml O₂/kg, and at grade of 25 and as SSWS of 51 meters/minute the work load is about 27.5 ml O₂/kg, or about 8 METs.

Thus, the SSWS and grade of 25 is a sufficient workload for many patients with congestive heart failure (CHF), and for cardiac patients and for many healthy elderly persons, and does not require running. This work range may also be sufficient as a submaximal test for patients with recent myocardial infarction or other patients where the doctor wants to exercise caution in limiting the workload. It can be seen that the workload 220 is not linear. Thus, levels selected should be equal workload steps or the time spent at different levels can be adjusted so that the work rate increase will be linear over the test protocol.

At the lower end of the work scale the chart gives a speed of about 84 meters per minute, or about 3.1 MPH on a level surface and gives a work load of about 3 METs. This work load may be used for submaximal testing, which can then be followed by ramping along the speed/slope line. Walking is most efficient for most humans above the age of 9 at speeds from 66 to 75 meters/minute, or at speeds of 2.4 to 2.8 miles per hour, but most adults are more comfortable at a slightly higher speed of around 83 meter/minute or 3.1 mph. These slower walking speeds may be used to give a lower work load, but little may be gained by walking speeds which become unnaturally slow. A walking speed at 75 meters/minute on a flat surface may be used to give a work load of about 9.5 ml O₂/kg.

Thus, lower speeds than shown on the chart may be used as a part of this protocol. A downward slope may also be used with this protocol, as maximal walking efficiency occurs with a down slope with a grade about negative 10. However, this is not an option generally included on current treadmills.

Younger children are more efficient at about 55 meters/minute or about 2 mph. Elderly, obese and infirmed subjects tend to walk closer to their maximum efficiency speed, which as indicated above is somewhat slower than the self selected walking speed for most adults.

At rest, a person's energy expenditure is 1 MET. Tests of patients with CHF at rest standing give a \dot{V}O₂ of approximately 7 ml O₂, or about 2 METs. Walking becomes less efficient at a rate of less than 66 meters per second, the approximate speed of maximal efficiency at a grade of zero, gives an estimated workload of about 2.5 METs. For Rickli's CHF patients referred for heart transplant (Rickli 2003) the average peak O₂ consumption among the group he identified as high risk was 17.5 (+/−4.9), or a work load of 5 METs. Thus even among this severely impaired group, a beginning walking speed of 66 meters per minute (about 2.5 mph), which is about the minimum natural walking speed for adults, is a sufficiently low workload to begin testing. In this invention it is recommended that the exercise test should begin with a walking speed not lower than 66 meters per second for most adult subjects. The slope of workload should increase from the beginning workload level to the predicted maximal workload level over the recommended work time, and then if the subject works past that level, the workload continues to be increased at the same rate until the test is terminated.

The work load may be raised by increasing the slope beyond a grade of 25 percent and by increasing the speed from the approximate 51 meters/min (1.9 mph) shown on the graphs in FIGS. 1 and 2. The increase in work rate is fairly linear to a slope of about 40%, but most treadmills do not go above a slope of 25%. The alternate is to increase the speed. This rate may be progressively increased for subjects who require a higher workload, such as younger and more athletic subjects.

During ambulation, 60 to 70 percent of the mechanical energy is conserved step to step by the inverted pendulum action of ambulation. Speeds which are too fast or too slow for the pendulum action become less efficient and less comfortable. Running becomes efficient for different reasons. Gravitational and kinetic energy is stored as the elasticity in the muscles and tendons and recovered with each step.

On level surfaces comfortable and efficient walking speeds do not overlap with comfortable and efficient running speeds. The speeds where neither walking nor running are efficient on level ground is referred to as the “run-walk gap”. FIG. 3 illustrates the run-walk gap. The grade of the slope is illustrated on the X axis and the speed in meters per minute on the Y axis. Dashed line 310 shows the minimum comfortable running speed and solid line 320 shows a maximum walking speed for a healthy young adult.

While self selected comfortable walking speeds are very similar for most adults, maximum walking speed decreases with age as muscle strength is lost, and is dependent on stature. Thus the run-walk gap increases with age and infirmity.

As slope increases, the walk-run gap narrows and there is little if any gap at a grade of over 20%, so there is little if any awkward speed gap at this grade as there is when going from a walk to a jog on a level surface. The walking efficiency and running efficiency (in joules per meter per second) are very similar at a slope of 25%, and the O₂ cost is similar whether the subject is walking or running at this slope for a given speed. This is likely due to the loss of the pendulum motion of walking at this slope. Thus, as the speed increases at this slope, the point where the subject converts from a walk to a run does not significantly alter the subjects metabolic cost. Thus the transition from walking to running at this slope has little effect on the work load, and the speed can be increased to smoothly increase the workload. This allows the subject to self select when the transition from a walk to a run occurs. This similarity in work load for walking and running at a 25 percent grade is not the case at lower inclines where walking and jogging have different energy costs.

Walking speeds between SSWS and the maximum walking speed may also be used to increase workload in subjects which require a workload than would be produced using the SSWS. As grade increases the walk-run gap narrows. The shift from SSWS to a higher speed closer to the maximum walking speed may be done progressively as the slope increases towards a grade of about 25 percent.

FIG. 4 shows the minimal running speed as a dashed line 410 and the self selected walking speed as the solid line 420. Also shown the speeds and slope steps of the Bruce Protocol as triangles 430. It can be seen that few of the steps in the Bruce Protocol correspond to self selected speeds.

The last 4 stages of the Bruce Protocol are appropriate speed/gradient combinations, however the lowest of these four stages gives a workload of about 13.5 METs, which is well above expected in most sedentary healthy adults. Thus it can be seen that only a single stage in the Bruce Protocol lies within a range of efficient ambulatory speeds which would be self selected by a typical middle aged person, and thus this protocol is ill-suited for most of the subjects who would undergo exercise testing as a medical test. Even for athletes, the higher Bruce protocol stages do not correspond to self selected running speeds.

The workload line shown in FIGS. 1 and 2 are meant to serve as an example, and may it be raised or lowered without changing the intent of this patent. What has been shown is an exemplary graph of speed/slope relations which most persons find comfortable. For example, younger and taller persons may prefer a quicker pace at a given slope, while smaller and older persons may prefer slower rates for a given slope. Young children may also be tested using the same method while using slower walking speeds.

There is little difference in comfortable walking speed between lean and obese patients (Browning 2005), but the comfortable pace does decline with age (Bohannon, Browning 2005, Malatesa 2002) and is affected by stature and gender (Bohannon 1997). These changes are likely secondary to differences in muscular strength, and with decline in strength, people tend to slow towards a more efficient walking speed. Thus, adjustment in comfortable walking speed may be used to accommodate the decline in the comfortable pace which occurs with age. Adjustments may also be made for gender, stature and muscular strength. Efficiency also declines with age (Maletesa 2003) and this too may be factored into the exercise protocol.

Running similarly has a most efficient and most comfortable minimum speed for a given slope. A running protocol using ISSP may be used, which may be advantageous for testing athletes and in sports medicine.

In FIG. 2 workload 220 shows typical self-selected walking speed for that grade. It can be seen that there is a non-linear relationship. In order to linearize the workload the time spent at each grade may be modified so that the workload increase is linear over time. This has advantages when the steps in the grade are limited to whole number grade steps. For example, if there are no partial grade increments, the number of steps is limited to the number of grades available. The speed may also be ramped within each step, but this becomes difficult as the speed may need to then decrease at the beginning of some steps in grade.

The workload may also be linearized by using smaller steps in grade, and increasing the grade in small steps so that the increase in workload is continuous. A combination of controlling the increments in grade and increments in time may also be used to linearize the increase in workload.

Ideally, the sub-anaerobic threshold work rate should be somewhat below the Δ\dot{V}O₂/ΔWR slope, and continue this incline of work rate throughout the test. This allows for the patient to achieve their peak workload without accumulating an increasing O₂ deficit. Patients with ischemic heart disease would fall below their sub-anaerobic slope when at their ischemic threshold and this information may be useful to distinguish patients with ischemic heart disease.

For most patients with a predicted \dot{V}O₂ₘₐₓ of under 35 mlO₂·kg⁻·min⁻¹, (10 METS) a 10 minute test is sufficient to give good results, and a minimum of 8 minutes to give good reliability for a \dot{V}O2 Max. For athletes and others with higher \dot{V}O₂ₘₐₓ, a longer duration of exercise may be required.

The workload velocity/slope equations may also be used for patients in cardiac/pulmonary rehabilitation. In rehabilitation, patients are typically prescribed a work load at 65 to 85% or their maximal workload. Another prescription may be to have the patient work at just below their anaerobic (AT) or ischemic threshold (IT) in the case of patients with known CAD. Data from the cardiopulmonary stress test may be used to find the prescribed level. After testing, a prescription for work can be based on testing and a combination of the speed/gradient and the time to slope to this gradient may be used for rehabilitation. Heart rate during the stress test where the event occurred could also be used to monitor the person during exercise and used to create the prescription. By using the patient's slope of increase to ramp to the desired exercise workload for the subject, the subject is less likely to have a significant O₂ deficit and less likely to experience ischemic, arrhythmic or other adverse events from the exercise. Using an appropriate and comfortable ramp over sufficient time should aid in giving a successful rehabilitation program.

As the subject trains, the Δ\dot{V}O₂/ΔWR slope may increase as the subject improves. The patient's rehabilitation prescription should follow this improvement with a progressive increase in workload.

Cool down periods are not usually used with maximal exercise testing when used as stress testing to try to reveal possible cardiac disease, as the sudden cessation of exercise gives an opportunity to watch for arrhythmias. Cool down periods may be used in patients with high risk, where the physician prefers to lower this risk. During exercise rehabilitation provocation of arrhythmias should be avoided and thus cool down periods are recommended and are typically used. Reversing the inverse speed/slope relationship may be done during the cool down phase. The rate at which the cool down phase workload declines may be user selectable and does not need to be linear. For example, it may be desirable to decrease the workload during the cool down phase to the below the anaerobic threshold or below the ischemic threshold over a few seconds, and then to continue decreasing the workload using the inverse speed/slope protocol over several minutes.

Lower Extremity Ergometry

Lower extremity (LE) ergometry is the most common exercise machine for cardiopulmonary testing in Europe. It has the advantage that it takes less space and is typically less expensive. It may also be more appropriate for some patients. In studies comparing LE ergometry and treadmill testing the peak work load and \dot{V}O₂ₘₐₓ is usually less with LE ergometry.

Ergometry is typically measured in mechanical power output in watts. This is a measure of the power needed to overcome the resistance of the machine. For example if the ergometer was an efficient generator, it would be producing that many watts of energy. Watts of output are not equivalent to the metabolic cost of exercise measured in watts. The conversion of muscular work and body mechanics to mechanical power has efficiency from about 22 to 27 percent depending mostly on pedaling speed, and crank length. The metabolic costs also include the basal metabolic costs.

An elite cyclist can produce about 400 watts with an efficiency of about 23.5%. A sedentary healthy adult should be able to produce from about 100 to 200 watts of power output while many patients with CHF would be expected to produce less than 100 watts of power.

Since efficiency of power output in watts can be roughly estimated at about 25%, it can be seen that the metabolic cost is about 4 times higher than the output. Thus for a patient with CHF a metabolic cost would be up to about 400 watts, and for a healthy sedentary adult the metabolic costs would be up to about 800 watts.

Since efficiency is dependent on pedal speed the subject should be encouraged to keep a cycling rate near the peak efficiency level for the workload they are working at. Commonly a metronome is set at a given pace to encourage the patient to maintain a given speed. If a metronome is used, it is usually set at 60 or 80 beats a minute. Most electronically braked LE ergometers will adjust resistance based upon cycling rate. Also, most LE ergometers also provide a digital RPM output.

McDaniel found that about 95% of the metabolic energy cost of cycling could be directly related to mechanical load, and less than 5% of the cost was determined by cycling speed or pedaling rate. Pedal speed was found to correlate slightly more closely with work than did pedal rate.
Pedal speed may be calculates as: (Pedal speed (m/s)=crank length (m)×pedaling rate (rpm)×2π/60)

Since the principal energy cost is the metabolic cost of work, subject comfort should be a main determinant in the decision of pedaling speed or rate. At heavy workloads the LE ergometer may be hard on the subject's knees. A fast pedaling pace will cause less strain than a slower pace rate with the same workload, and be more comfortable especially for persons with arthritis.

McDaniel also showed that at low power output, metabolic cost is strongly influenced by the cost of unloaded cycling. His group found that the cost of unloaded cycling increased with pedal speed and ranged from a low of around 73 metabolic watts at 0.91 meters/second (the lowest pedal speed tested; 40 cycles per minute crank length 145 mm) to around 297 metabolic watts at 2.04 meters/second, the highest pedal speed tested (pedaling rate of 100/minute crank length 195 mm). Since a significant metabolic load may be placed with unloaded cycling, we teach the use of limiting the mechanical resistance of ergometers while using variable cycling speeds to contribute to the workload placed on the subject especially for subjects with a low predicted \dot{V}O₂ₘₐₓ or those undergoing submaximal testing.

Cost of cycling can be described by the cost of unloaded cycling plus the metabolic costs from mechanical resistance. The metabolic costs are equivalent to approximately 4 times the mechanical power output of the ergometer. The predicted peak work load for many patients with CHF is within the workload produced by unloaded cycling. A ramping protocol for exercise testing using ergometry may use unloaded LE or arm ergometry, or minimally loaded ergometry in many of these subjects, and would also be sufficient for many patients for submaximal exercise tests. The ramping protocol may slowly increase the load and cycling speed over the selected exercise period (see above) to achieve the expected work load.

FIG. 5 chart shows a representative workload on watts of metabolic costs with an unloaded ergometer and a low resistance workload. The metabolic cost is given on the Y-Axis and the pedal speed on the X-axis. Solid line 510 shows the unloaded work load. Dashed line 520 shows the workload when the unloaded workload is supplemented with an additional zero to 92 watts of work. This gives a total workload with might be used for a subject with CHF. Pedaling speed depends on the crank length of the ergometer being used, and would not be altered during the protocol. The workload depends on the subject matching the speed given by the metronome. The metronome may be controlled by a microprocessor or computer and accelerate during the test. Workload may be linearized by adjusting the amount of time at each work level. Since resistance is fairly linear, as resistance is added the workload becomes less curvilinear and would need less adjustment to linearize it.

Arm ergometry is sometimes used for patients in wheelchairs or for patients who otherwise are unable to walk on a treadmill or use a bicycle ergometer. The methods disclosed herein for bicycle ergometry are also applicable for arm ergometry and may be adapted for that use.

The ramping of workload should ideally not exceed, nor be much less than the subject's Δ\dot{V}O₂/ΔWR. The typical healthy patients can increase their workload at a rate of approximately 10.2 mlO2/min/watt.

The formula:
Δ\dot{V}O₂/ΔWR=(Peak \dot{V}O2−Resting \dot{V}O2)/[(T−0.75)×Slope]
may be used to calculate Δ\dot{V}O₂/ΔWR. Slope is the increase in mechanical work load in watts, and T is time in minutes. This may be rewritten in order to give the desired slope of work load as follows:
Slope=(Predicted \dot{V}O2−Resting \dot{V}O2)/[(T−0.75)×predicted Δ\dot{V}O₂/ΔWR]
For example, a normal individual with an expected value for an Δ\dot{V}O₂/ΔWR of 10.2, a predicted work increase of 2480 ml O2 (Predicted \dot{V}O2−Resting \dot{V}O2), and a time of 10 minutes, slope is calculated to be
2480 ml O2/[9.25×10.2]=24.9 watts per minute.
This reflects the mechanical workload, and can to be converted to metabolic workload by estimating the predicted efficiency of the subject. Using a typical value of around 25% efficiency, the metabolic slope could be estimated to be approximately 100 watts per minute. As with treadmill testing, the rate of increase of workload may be controlled by data from physiologic measurements such as heart rate or the \dot{V}O₂ as disclosed later herein.

The workload may also be predicted from nomograms or equations taking into account the subject's age, gender, level of fitness and other metrics. The ergometry protocol may be set to have the subject reach their predicted peak workload over a time period which allows them to best reach this workload. Once the predicted workload has been determined the contribution from pedaling and form resistance (output power) can be determined and then be set based on metabolic cost rather than metabolic output power. In this invention we teach the use of “the cost of pedaling” to determine output power. This may be used to decrease stress on the joints and increase comfort for the subject. If the subject exceeds the predicted workload, the pedaling speed and or the workload power output demand may be increased until the test is completed.

If the subject does not match the metronome speed the ergometer should compensate the output power demand based on the metabolic cost of work (the cost of pedaling and the power output).

This invention may use algorithms wherein
a. Subject data (such as but not limited to: age, gender, height, weight, medications, and exercise tolerance, health or disease status) are used to calculate a predicted peak workload.
b. This predicted peak workload will be spread over a time period. This time period should be at least 8 minutes, but is preferably 10 to 17 minutes. It should be longer for athletic persons whose predicted workload is greater than 35 mlO₂·kg⁻¹·min⁻¹ (10 METs).
c. The prescribed test duration may be determined by a second algorithm.
d. The exercise machine may be controlled by use of a microprocessor or computer to ramp the workload continuously or as a series of small steps at a rate to meet the predicted peak workload over the prescribed time period. The rate of increase may be controlled so that the physiologic workload rate increase is linearized.
e. If the subject is able to continue beyond the prescribed time period, the workload continues to increase at the same rate as before the end of the prescribed time period until the test is terminated (either by the subject or the tester).
f. The rate of increase in workload may preferably be set so that it does not exceed the Δ\dot{V}O₂/ΔWR slope during the first few minutes of exercise. The Δ\dot{V}O₂/ΔWR slope may be determined through data from the collection of respiratory gas during exercise.
g. A variable speed metronome may be used with the ergometer. The workload can be calculated in real time from a combination of the free wheeling work plus the resistance work. The metronome can set a pace which is meant to be most appropriate for the subject.
h. The rate of increase in workload may be controlled using feedback from physiologic measurements such as the heart rate, the \dot{V}O₂ or the respiratory exchange ratio (RER).
   These algorithms may be used in the form of a computer program. These protocols are designed to give accurate workloads throughout the test and may be used with submaximal tests.

Treadmill Considerations

If during a treadmill test the subject leans on the stabilization bars during exercise, the transfer of weight will decrease the work load of the subject, and may give incorrect test results. This can be accommodated by factoring in the use of hand rails into the protocol, but this only poorly predicts how much the workload has been altered. Many doctors and technicians who operate the stress test equipment are unaware that the use of the hand rails alters the test results.

One of the principal reasons for having and using rails is to help the subject with stability, and adapt to speed. The subject is more likely to use the rails if the speed is uncomfortable, or if the speed makes it difficult to keep pace. Use of the treadmill protocol described herein should make the walking or running speeds more natural for the subjects, so that they are less likely to feel the need to stabilize themselves, or to pace themselves to the machines speed.

The treadmill may also be designed to prevent transfer of weight from the subject to the treadmill rails. One method for this is to use a stabilization method which does not allow the transfer of weight from the subject. FIG. 6 illustrates various embodiments of hand holds 610 and 630 for treadmills which avoid transfer of weight to the hand hold. Hand holds 610 is attached to “tow rope” designs 620 similar to that used in water skiing, while hand hold 630 is connected by flexible cords 640 and 645 attached to two fixation points 646 and 647. These designs allow the subject to maintain speed but do not allow weight offset. A stop switch may be included into the hand hold so that the subject can stop the treadmill if need be.

FIG. 6 also illustrates this method showing a simplified view of a treadmill 670. No rails are shown in order to simplify the illustration. Pivot bar 650 is illustrated with a single pivot point although it may have two or more points of attachment. In this design a hand hold is attached to a hinged mechanism 660. This design can allow a switching mechanism to slow or stop the tread mill if the bar is pulled down too far, and can act as a safety feature for the treadmill. Again in this design the subject can use the hand hold to match the treadmill speed, but it will not allow transfer of weight to the bar.

Physiologic Ramping

Several formulas have been published for estimating the \dot{V}O₂ₘₐₓ for subjects who will have a cardiopulmonary exercise test. These are useful in setting the predicted workload, which can then be spread over the prescribed duration of the test. An individual may however not respond as expected. The physiologic response to exercise may be used during the test to control the ramping of the work load.

One way to use physiologic response for controlling the ramping of work load during exercise is to use the heart rate during exercise. Heart rate generally is linearly related to work rate during exercise. A subject's maximum heart rate may be predicted from the subject's age or age and gender, health status, and medication use, such as beta-blockers. Ideally the heart rate should increase steadily throughout the exercise test as the work load increases. If the heart rate increase is faster than expected, it may mean that the ramping speed has been set too high and that the subject would reach their maximal work load early, which could give unreliable test results. Thus, the heart rate may be used to control the ramping of the work rate.

FIG. 7 illustrates a simplified example. The heart rate is shown in the Y-Axis, and the exercise time along the X axis. Asterisks 720 mark the heart rate at rest and during the first minutes of exercise. Vertical line 710 marks the onset of exercise. Dashed vertical line 730 shows the prescribed test duration. Horizontal dashed line 740 shows the predicted heart of 172 for this example. Solid line slope 750, shows the expected heart rate slope given the heart rate at the beginning of exercise, the predicted maximum heart rate, and the targeted duration of the test. Asterisks 720 at the beginning of exercise form a slope which if continued as shown in line 770 would reach the predicted heart rate after only about 6 minutes of exercise which would likely be insufficient to allow the subject to attain their \dot{V}O₂ₘₐₓ.

Several methods may be used to reset the work rate. A simple method is illustrated. Point 780 illustrates where the projected time that heart rate would reach its predicted maximum. For purpose of example, assume that the predicted maximum work rate for the subject had been 120 watts, and the targeted test duration 10 minutes. The heart rate slope at the beginning of exercise 770 suggests that the maximum heart rate would be reached in about 6 minutes as shown by point 780. A new target work load may be calculated as 120 watts×6/10, or 72 watts. Thus, the rise in heart rate suggests that this subject's maximum heart rate will be reached with a work load of 72 watts, and the target workload may be adjusted to this new workload level to be achieve over the remaining portion of the target time. Dashed line 760 shows a new predicted slope for heart rate increase.

Physiologic measures during exercise may also be used to control the rate of workload changes when it is found that physiologic measurements are increasing more rapidly or slowly than expected. However, a test which is a few minutes too short and has a work rate increase too steep is much more likely to compromise results than a test which is a few minutes longer than required and has a slower work rate ramp. An impaired response of heart rate to work load may also be a sign of disease rather than of athleticism, and accelerating the workload would not be helpful in the situation. Predicted workloads are based on a wide range of subjects including athletes. There is a low likelihood of an under estimated workload for a healthy person and little risk associated with a slower rise in workload. There is much higher likelihood and higher risk associated with an over estimated workload for an individual who may have an undiagnosed and asymptomatic condition.

Respiratory data may also be used to adjust the work rate ramp during an exercise test. Some respiratory variables may include but not limited to the \dot{V}O2, \dot{V}CO2, \dot{V}E, or a combination of this data, such as the RER. Another variation to control the work rate increase is by limiting the O₂ deficit.

Like heart rate, the \dot{V}O₂ generally is linearly related to workload during exercise. A subject's \dot{V}O₂ₘₐₓ may be predicted from various algorithms which may include the subject's age, gender, body mass, muscle mass, obesity, health and training status, and medication use and other variables. Ideally the \dot{V}O₂ should increase steadily throughout the exercise test as the work load increases. If the \dot{V}O₂ increase is faster than expected, it may mean that the ramping speed has been set too high and that the subject would reach their maximal work load early, which could give unreliable test results. Thus, the \dot{V}O₂ may be used to control the ramping of the work rate similar to the method described above for heart rate.

Another example a physiologic measure that can be used for controlling the ramp of workload is the RER. The RER is the ratio of expired CO2 to the expired O2. At rest this ratio is typically at about 0.80. During exercise this ratio increases. The anaerobic threshold, (AT) is a point which the consumption of oxygen is insufficient for the metabolic needs of the muscular activity, and glucose is anaerobically metabolized in part through the lactic acid cycle.

The AT typically occurs at between about 46 and 68% of \dot{V}O₂ₘₐₓ, depending on several factors including age, gender, and muscle mass. The lower limit of normal has less variance than the upper limit, and the AT to \dot{V}O₂ₘₐₓ ratio. AT to \dot{V}O₂ₘₐₓ ratios lower than the lower limit of normal are often associated with disease states. An RER of 1.0 may be used as an estimate of the AT during an exercise test.

For the example of a 40 year old man with an AT to \dot{V}O₂ₘₐₓ ratio lower limit of 44%, the RER would not be expected to rise to 1.0 until 44% of the test work load has been met or 44% of time length passed if the time length is adequate. Thus in an exercise test in which the goal is to exercise to \dot{V}O₂ₘₐₓ incrementally over ten minutes of increasing workload, the AT should not be achieved until a minimum of 4 minutes and 24 seconds graded exercise has been accomplished. This can be used in testing where the RER is assessed in real time or near real time. This information may be used to lower the workload slope in a test where the RER rises faster than a predicted slope for the lower limit of normal AT to \dot{V}O₂ₘₐₓ ratio over test prescribed test time.

By measurement of the RER at the beginning of exercise, the difference between that and 1.00 may be calculated, and thus a predicted slope maybe calculated which will give an RQ of 1.00 only after the percent of the expected duration of exercise for the lower limit of normal for the AT to \dot{V}O₂ₘₐₓ ratio. If the real time slope of the RER or the RER significantly exceeds the predicted slope or predicted RER during the first minutes of exercise prior to the AT, it may indicate that the work rate increase is too high for the patient and the rate of increase in workload may be slowed. A new work rate increase slope may then be calculated for the remainder of the test.

It can be appreciated that if the work rate slope is lowered and the target time for the test remains the same, that the workload at the end of the target time will be lower than had been originally predicted.

Multiple variations of this concept may be utilized within the scope of this invention. FIG. 6 shows slope being calculated once after about 2 minutes. The slope may be recalculated multiple times during exercise. The workload is not shown in FIG. 6. The work rate slope may be increased or decreased according to the physiologic response.

Other physiologic responses may also be used for controlling exercise rate and workload increases. These include but are not limited to respiratory rate, respiratory volume, respiratory gases and cardiac output, or combinations and derivatives of these and other variables.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed, but that modifications and other embodiments are intended to be included within the scope of the appended claims. For example, although the embodiment shown in FIG. 1 shows a rate/slope relationship for a typical adult, this rate slope relationship may be adjusted for children, elderly, infirmed, or other individuals who are not typical of healthy adults without violating the intent of this patent.

Multiple variations of ways to adjust work rate slopes by use of physiologic measures of heart rate or respiratory gases are readily apparent to one skilled in the art. Simplified examples are given for purpose of explanation only and not meant to limit the method disclosed here.

Another example is sub-maximal exercise tests, which do not require the subject to reach their \dot{V}O₂ₘₐₓ, but appropriate ramping of workload and a comfortable test protocols remain important. Where these inventions apply to submaximal exercise testing as well as to maximal tests. \dot{V}

Although specific terms are employed herein, they are used in a generic and descriptive sense only and not for purposes of limitation.

Various aspects of disclosure:
1. A treadmill exercise protocol during which speed decreases as the slope increases in at least during part of the exercise test.
2. The device in aspect 1 where the combination of grade and speed are set at levels which are natural speeds for ambulation for the given slope.
3. A treadmill exercise protocol which uses natural walking speeds at various slope between a negative grade of 11% to a positive grade of 25% to achieve desired exercise loads.
4. The exercise protocol in aspect 3 where higher work loads are achieved by increasing the treadmill speed only after a grade of 20% or above has been reached.
5. The use of physiologic response to exercise to control the work rate during exercise.
6. The method of controlling exercise workload in aspect 5 where the physiologic response is heart rate, cardiac output, respiratory rate, respiratory volume, expired respiratory gases, the ratio of exhaled respiratory gases or a combination of these.
7. The methods of using measurements of the physiologic response to exercise to control the workload during exercise for exercise testing, training, and rehabilitation.
8. The method in aspect 7 where once a target exercise workload is achieved the subject continues to work at that prescribed level.
9. A cool down period for a treadmill exercise protocol during which speed increases as the slope decreases in at least during part of the cool down exercise test.
10. An exercise protocol using guided variable speed unloaded cycle ergometry for exercise testing or training, or use of loaded cycle ergometry in combination with variable guided speed cycle ergometry for exercise testing or exercise training.
11. Use of a variable metronome for guiding exercise speed based on the desired workload of the exercise.
12. The use of hand holds with a treadmill which prevent the transfer of weight from the user to the hand holds during normal exercise.
13. The method of calculating a targeted maximum exercise work load during an exercise test from physiologic response to exercise.
14. The method in aspect 13 where the calculated maximum exercise workload is distributed over the remaining targeted test duration.

1. A method of exercising a subject, comprising the steps of:
providing a treadmill, the treadmill having an adjustable speed and an adjustable slope; and
providing a protocol for use with the treadmill, the protocol being configured to test a subject using the subject's substantially natural cadence at different workloads.

2. A method of exercising a subject, comprising the steps of:
providing a treadmill, the treadmill having an adjustable speed and an adjustable slope; and
providing a protocol for use with the treadmill, the protocol being configured to increase the subject's workload by decreasing the speed of the treadmill while increasing the slope of the treadmill during at least a portion of the exercise.

3. A method of exercising a subject, comprising the steps of:
providing an exercise device, the exercise device having a variable speed and at least one of a variable slope and a variable resistance;
determining a physiologic response of a subject using the exercise device;
providing a protocol for use with the exercise device, the protocol being configured to control the workload of a subject based on a physiologic response of the subject; and
varying at least one of the speed, the slope and the resistance of the exercise device based on the physiologic response of the subject.

4. The method of claim 3, wherein the physiologic response includes at least one of heart rate, cardiac output, respiratory rate, respiratory volume, expired respiratory gasses and the ratio of exhaled respiratory gasses.

5. A method of exercising a subject, comprising the steps of:
providing a cycle ergometer, the cycle ergometer having a variable resistance and a variable cycling speed;
providing a protocol for use with the cycle ergometer, the protocol being configured to increase the subject's workload by increasing the cycling speed resistance throughout a portion of the exercise.

6. An accessory for use with a treadmill, the accessory comprising:
at least one hand hold configured to be grasped by a user;
an attachment structure disposed in mechanical cooperation with the at least one hand hold and configured to be operatively coupled to a portion of a treadmill; and
wherein the hand hold is substantially unsupported in the vertical direction.

7. The method of claim 1, wherein the protocol is configured to adjust the speed of the treadmill based on the subject's substantially natural cadence at a given slope of the treadmill.

8. The method of claim 1, wherein the protocol is configured to adjust the speed of the treadmill based on a natural speed for ambulation at the particular slope of the treadmill.

9. The method of claim 1, further including increasing the workload by increasing the speed of the treadmill after the slope is at least about 20%.

10. The method of claim 2, further including increasing the workload by increasing the speed of the treadmill after the slope is at least about 20%.

11. The method of claim 1, wherein the protocol is configured to decrease the subject's workload by increasing the speed of the treadmill and decreasing the slope of the treadmill during at least a portion of the exercise.

12. The method of claim 3, further including varying the workload of the subject based on the physiologic response of the subject while allowing the subject to exercise with a substantially natural cadence.

13. The method of claim 3, further including the step of using the physiologic response of the subject to help determine a target workload of a subject.

14. The method of claim 13, further including the step of varying at least one of the speed, the slope, and the resistance of the exercise device to help a subject reach the target workload of the subject over a selected time interval.

15. The method of claim 3, further including the step of estimating a maximum workload for the subject based on the physiologic response of the subject.

16. The method of claim 15, further including the step of varying at least one of the speed, the slope and the resistance of the exercise device such that the slope of the workload increases the workload of the subject to the estimated maximum workload of the subject and is substantially evenly distributed over the remainder of a predetermined duration of the exercise.

17. The method of claim 16, further including exercising the subject at a given workload slope beyond the predetermined duration of exercise until the subject has reached a maximum workload.

18. The method of claim 5, further including the step of providing a metronome to help guide the subject's cycling speed.

19. The method of claim 1, wherein the protocol is configured to increase the subject's workload at a substantially linear rate over a selected time.

20. The method of claim 2, wherein the protocol is configured to increase the subject's workload at a substantially linear rate over a selected time.

21. The method of claim 5, wherein the protocol is configured to increase the subject's workload at a substantially linear rate over a selected time.

22. The method of claim 5, further including varying the resistance of the cycle ergometer during exercise such that the subject's workload reaches a target amount.

23. The method of claim 5, wherein the cycle ergometer is unloaded.

24. The method of claim 2, wherein the protocol is configured to decrease the subject's workload by increasing the speed of the treadmill and decreasing the slope of the treadmill during at least a portion of the exercise.

25. The method of claim 3, wherein the exercise device is a cycle ergometer having a variable cycling rate, and further including varying the cycling rate to vary the workload of the subject.
